(19) United States
(12) Patent Application Publication
(10) Pub. No.: US 2025/0235123 A1
(43) Pub. Date: Jul. 24, 2025

(54) METHOD AND SYSTEM FOR HEALTH SURVEILLANCE USING WI-FI TO QUANTIFY GAIT PARAMETER

(71) Applicant: National Cheng Kung University, Tainan (TW)

(72) Inventors: Chi-Lun LIN, Tainan (TW); Shih-Yuan CHEN, Tainan (TW); Tzu-Yang WENG, Tainan (TW); Chun-Hsiang Chang, Tainan (TW); Chi-Hung HO, Tainan (TW)

(21) Appl. No.: 18/966,561

(22) Filed: Dec. 3, 2024

Related U.S. Application Data
(60) Provisional application No. 63/623,792, filed on Jan. 22, 2024.

(51) Int. Cl. A61B 5/11; A61B 5/00; A61B 5/05

(52) U.S. Cl. CPC A61B 5/112; A61B 5/05; A61B 5/1126; A61B 5/7203; A61B 5/7257; A61B 2560/0223

(57) ABSTRACT

The present invention relates to a method for health surveillance using Wi-Fi to quantify gait parameter, comprising: (S1) establishing a Wi-Fi space by a transmitter and a receiver, wherein the transmitter is used for emitting a first wireless signal; (S2) allowing a human body to move in the Wi-Fi space, receiving a second wireless signal by the receiver, and extracting a CSI signal from the second wireless signal; (S3) preprocessing the CSI signal, thereby obtaining a denoised CSI signal; (S4) performing a feature extraction on the denoised CSI signal, thereby obtaining a human body CSI gait parameter; and (S5) calibrating the human body CSI gait parameter using a calibration equation, thereby obtaining a calibrated gait parameter.

### FIELD OF THE INVENTION

The present invention relates to a method for quantifying a gait, especially referring to a method for accurately quantifying a gait parameter of a human body by using Wi-Fi.

### BACKGROUND OF THE INVENTION

When measuring a movement parameter of a human body of a subject during the medical research, the subject will be equipped with a wearing device, and the movement parameter or a limb movement of the human body will be obtained by an inertia measuring unit or a camara device respectively to evaluate whether an abnormal phenomenon exists in the movement of human body. However, the wearing device interferes the extension of human limbs to a certain degree and compresses the human body for long-term wearing, making it unsuitable for every patient. Moreover, the camara device, limited by the requirement of shooting angle and the privacy problem, is not suitable for long-term tracking and measuring.

In view of the problem aforementioned, current relevant research indicates that a gait parameter can be measured through Wi-Fi in the environment. However, error exist in the results measured using Wi-Fi with respect to the actual results of the human body movement, resulting in a human body healthy surveillance cannot be performed accurately. In summary, a method for accurately quantifying the gait parameter of the human body by using Wi-Fi is needed nowadays.

### SUMMARY OF THE INVENTION

The problem that the present invention aims to solve comprises: providing a method for accurately quantifying a gait parameter of a human body by using Wi-Fi.

To accomplish the purpose, the present invention provides a method for health surveillance using Wi-Fi to quantify gait parameter, comprising: (S1) using a transmitter and a receiver to establish a Wi-Fi space, wherein the transmitter is configured to emit a first wireless signal; (S2) moving a human body in the Wi-Fi space to receive a second wireless signal by the receiver, and extracting a CSI signal from the second wireless signal, wherein the second wireless signal is formed by the first wireless signal reflected from the human body; (S3) preprocessing the CSI signal to obtain a denoised CSI signal; (S4) performing a feature extraction on the denoised CSI signal to obtain a human body CSI gait parameter; and (S5) calibrating the human body CSI gait parameter using a calibration equation to obtain a calibrated gait parameter.

Preferably, in the step (S1), a line of sight A̅B̅ is formed between a point A on the transmitter and a point B on the receiver, and 5 m≥A̅B̅≥1 m; and in the step (S2) an angle θ is formed between a moving direction of the human body and a perpendicular bisector of the line of sight A̅B̅, and θ≤45°.

Preferably, wherein the step (S3) comprises: using a wave filter to filter a high frequency noise in the CSI signal, and performing a principal component analysis (PCA) on the CSI signal, thereby extracting a human body gait related signal from the CSI signal; or wherein the step (S4) comprises: performing a short-time Fourier transform (STFT) on the denoised CSI signal to obtain a spectrogram, and tracking a high energy region in the spectrogram to form a trunk velocity contour on the spectrogram thereby obtaining the human body CSI gait parameter.

Preferably, in the step (S5), a step for obtaining the calibration equation comprises: fitting the human body CSI gait parameter with a human body actual gait parameter, wherein a step for obtaining the human body actual gait parameter comprises: equipping a mark on a first body part of the human body and tracking the mark using a tracking software thereby obtaining a first actual walking velocity curve.

Preferably, in the step (S5), a step for obtaining the calibration equation comprises: fitting the human body CSI gait parameter with a human body actual gait parameter, and establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

The present invention further provides a system for health surveillance using Wi-Fi to quantify gait parameter, comprising: a transmitter configured to emit a first wireless signal; a receiver arranged relative to the transmitter to optionally receive a second wireless signal and extract a CSI signal from the second wireless signal, wherein the second wireless signal is formed by the first wireless signal reflected from a human body; and a processor arranged relative to the receiver and configured to receive the CSI signal and execute an instruction as the following: (SA) preprocessing the CSI signal to obtain a denoised CSI signal; (SB) performing a feature extraction on the denoised CSI signal to obtain a human body CSI gait parameter; and (SC) calibrating the human body CSI gait parameter to obtain a calibrated gait parameter using a calibration equation.

Preferably, a line of sight A̅B̅ is formed between a point A on the transmitter and a point B on the receiver, and 5 m≥A̅B̅≥1 m; and an angle θ is formed between a moving direction of the human body and a perpendicular bisector of the line of sight A̅B̅, and θ≤45°.

Preferably, wherein in the step (SA) comprises: using a wave filter to filter a high frequency noise in the CSI signal, and performing a principal component analysis (PCA) on the CSI signal, thereby extracting a human body gait related signal from the CSI signal; or wherein the step (SB) comprises: performing a short-time Fourier transform (STFT) on the denoised CSI signal to obtain a spectrogram, and tracking a high energy region in the spectrogram to form a trunk velocity contour on the spectrogram thereby obtaining the human body CSI gait parameter.

Preferably, wherein in the step (SC), a step for obtaining the calibration equation comprises: fitting the human body CSI gait parameter with a human body actual gait parameter, wherein a step for obtaining the human body actual gait parameter comprises: equipping a mark on a first body part of the human body and tracking the mark using a tracking software thereby obtaining a first actual walking velocity curve.

Preferably, in the step (SC), a step for obtaining the calibration equation comprises: fitting the human body CSI gait parameter with a human body actual gait parameter, and establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

The effectiveness of the present invention compared with prior art comprises: in prior art, there are gaps or differences between the human body gait measurement result measured by using Wi-Fi directly and the result of actual walking of the human body, therefore the Wi-Fi measurement lacks practical value and cannot be used to determine a healthy degree of a subject. The method provided by the present invention comprises analyzing the result of CSI signal and fitting them with the human body actual gait parameter to obtain the calibration equation, wherein the gait parameter is close to the human body actual gait parameter after the gait parameter of CSI signal is calibrated by the calibration equation, thereby can be used to analyze the health status of the human body. On the other hand, the present invention can analyze one or more time period of a human body gait change curve, calculate the gait parameter to track the history measuring result of the subject's gait, and remind the subject when abnormality occurs in their body, thereby improving the medication administration or the treatment strategy to the subject.

### BRIEF DESCRIPTION OF THE DRAWINGS

FIG. 1 is a flow diagram for illustrating the method for health surveillance by using Wi-Fi to quantify the gait parameter.

FIG. 2 is a picture diagram for representing the spectrogram processed by SFTF.

FIG. 3 is a picture diagram for representing the spectrogram having the highest energy curve.

FIG. 4 is a picture diagram for representing the spectrogram having the weighted average curve of the human body gait.

FIG. 5 is a plan diagram for illustrating the arrangement of the gait measurement and the environment conditions

FIG. 6 is a curve diagram for illustrating the correlation between the Tracker velocity curve and the CSI velocity curve before calibration.

FIG. 7 is an experiment result diagram for illustrating the correlation between the Tracker velocity curve and the calibrated CSI velocity curve by the linear regression.

FIG. 8 is a curve diagram for illustrating the relative correlation between the Tracker velocity curve and the calibrated CSI velocity curve.

FIG. 9A to 9B are bar diagrams for illustrating and comparing the velocity error and DTW before and after calibration.

FIG. 10A to 10B are bar diagrams for illustrating the affection of the velocity error and DTW under different variables after calibration.

FIG. 11 is a plan diagram for illustrating the arrangement of the device for gait measurement and the environment condition from one of embodiments.

FIG. 12 is a plan diagram for illustrating the arrangement of the device for gait measurement and the environment condition from another one of embodiments.

FIG. 13 is a plan diagram for illustrating the Fresnel zone theory.

FIG. 14A to 14B are a series of bar diagrams for illustrating the effect of the line of sight on the error of velocity measurement and the DTW.

FIG. 15A to 15B are a series of bar diagrams for illustrating the effect of walking deviation angle on the error of velocity measurement and the DTW.

FIG. 16 is a block diagram for illustrating the structure characteristics of the system for health surveillance by using Wi-Fi to quantify the gait parameter.

FIG. 17 is a flow diagram for illustrating the execution steps of the processor.

### DETAILED DESCRIPTION OF THE INVENTION

To elaborate the purpose, effectiveness, or characteristic of the present invention more properly, some preferred embodiments are provided as the following:

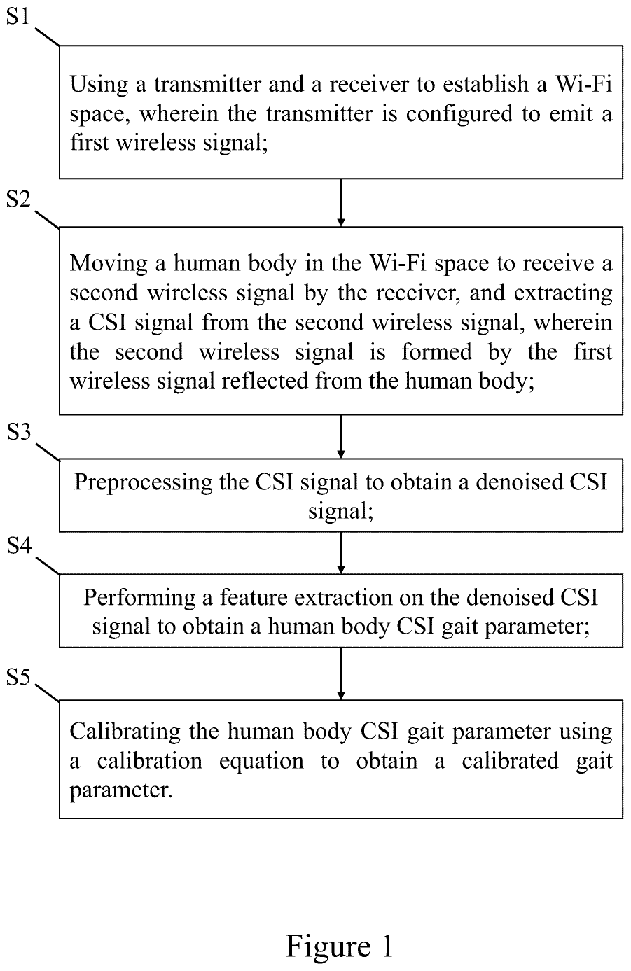

One of the aims of the present invention is to provide a method for health surveillance using Wi-Fi to quantify gait parameter, as shown in FIG. 1, comprising: (S1) using a transmitter and a receiver to establish a Wi-Fi space, wherein the transmitter is configured to emit a first wireless signal; (S2) moving a human body in the Wi-Fi space to receive a second wireless signal by the receiver, and extracting a CSI signal, wherein the second wireless signal is formed by the first wireless signal reflected from the human body; (S3) preprocessing the CSI signal to obtain a denoised CSI signal; (S4) performing a feature extraction on the denoised CSI signal to obtain a human body CSI gait parameter; and (S5) calibrating the human body CSI gait parameter using a calibration equation to obtain a calibrated gait parameter.

In some embodiments, in the step (S5), a step for obtaining the calibration equation comprises: fitting the human body CSI gait parameter with a human body actual gait parameter. Specifically, a condition that can be adjusted in the process of obtaining the human body CSI gait parameter or a human body actual gait parameter comprises: subject's age, sex, or body type; the hardware condition of, or the correlation between the transmitter and the receiver settings; the angle and the distance of a line started from an start point to an end point of walking of the subject, or the line relative to a line of sight (LoS); or step length, cadence and any combination thereof of the subject. In some preferred embodiments, the human body CSI gait parameter is a first velocity and time correlation diagram obtained by CSI measurement, from which a first human body velocity curve is formed, wherein a walking velocity of the human body V_{CSI} is obtained in accordance with the first human body velocity curve; and the human body actual gait parameter is a second velocity and time correlation diagram obtained by a wearing device or an image tracking system, from which a second human body velocity curve is formed, wherein a walking velocity of the human body V_{REAL}. In some preferred embodiments, the V_{CSI} can be calibrated to become a V_{correction} using the calibration equation, wherein the V_{correction} is close to V_{REAL}, in other words, the V_{correction} represents the actual walking velocity of the human body, and it will be further compared with a default threshold to determine whether a health status of the human body is qualified.

In some embodiments, the method further comprises: (S6) calculating T1 and T2 in one or more time period, and delivering a health surveillance report when T2>T1, wherein T1 is the total time in which the calibrated gait parameter (V_{correction}) of the subject is larger than the default threshold, T2 is the total time in which the calibrated gait parameter (V_{correction}) of the subject is smaller than the default threshold. Concretely, the default threshold is adjustable in accordance with the subject's age, sex, body weight, or health condition, T1 represents the total time during which the performance of the subject's gait is good, and T2 represents the total time during which the performance of the subject's gait is poor, in other words, through comparing the difference between T1 and T2, the gait performance of the subject can be evaluated, and according to the difference between T1 and T2, the health status of the subject can be evaluated for improving the medication administration or for changing treatment direction. In some embodiments, the one time period refers to, for example, 1 a.m. to 10 a.m.; the more time period refers to, for example, 1 a.m. to 2 a.m., 4 a.m. to 5 a.m., and 7 a.m. to 8 a.m.

In some embodiments, the method further comprises: (S7) calculating T1 and T2 in a first time period, wherein T1 is the total time in which the calibrated gait parameter (V_{correction}) of the subject is larger than the default threshold, T2 is the total time in which the calibrated gait parameter (V_{correction}) of the subject is smaller than the default threshold; calculating T3 and T4 in a second time period, wherein T3 is the total time in which the calibrated gait parameter (V_{correction}) of the subject is larger than the default threshold, T4 is the total time in which the calibrated gait parameter (V_{correction}) of the subject is smaller than the default threshold; and delivering a health surveillance report when

$$\frac{T 1}{T 1 + T 2} > \frac{T 3}{T 3 + T 4} ,$$

wherein the second time period is later than the first time period. Concretely, the improvement of gait of human body can be understood through analyzing the performance of the gait in different time period, that means, when

$$\frac{T 1}{T 1 + T 2}$$

is larger, the performance of the subject in the first time period is better, when

$$\frac{T 3}{T 3 + T 4}$$

is larger, the performance of the subject in the second time period is better, and conceivably, when

$$\frac{T 1}{T 1 + T 2} > \frac{T 3}{T 3 + T 4} ,$$

the performance of the subject in second time period is worse than the performance of the subject in the first time period, further condition tracking or medication improvement may be needed for preventing from health deterioration of the subject. In some embodiments, the length of total time of the first time period is identical to or is not identical to the length of the of total time of the second time period, and the first time period and the second time period can be single time period or multiple time periods respectively.

In some embodiments, in the step (S5), a step for obtaining the calibration equation comprises: fitting the human body CSI gait parameter with a human body actual gait parameter; and establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}. In some embodiments, the calibrated gait parameter comprises the calibrated human body walking velocity V_{correction}.

In some embodiments, to ensure the accuracy of the measurement, and to avoid the difference between the human body CSI gait parameter and the human body actual gait parameter is too large to calibrate, in the step (S1), a line of sight A̅B̅ is formed between a point A on the transmitter (1) and a point B on the receiver (2), and 5 m≥A̅B̅≥1 m; and in the step (S2) an angle θ is formed between a moving direction of the human body and a perpendicular bisector of the line of sight A̅B̅, and θ≤45°. In some embodiments, when human body moves along the perpendicular bisector of the line of sight A̅B̅, the error between the human body CSI gait parameter and the human body actual gait parameter can be minimized, in addition, the human body CSI gait parameter can be almost identical to the human body actual gait parameter after calibrated by the calibration equation.

In some embodiments, to avoid the interference of the noise, and to prevent the accuracy of the gait parameter measurement from affecting by the signal irrelevant to the gait of the human body, in the step (S3) comprises: using a wave filter to filter a high frequency noise in the CSI signal, and performing a principal component analysis (PCA) on the CSI signal, thereby extracting a human body gait related signal from the CSI signal; moreover, to quantify a gait parameter extracted from the denoised CSI signal, the step (S4) comprises: performing a short-time Fourier transform (STFT) on the denoised CSI signal to obtain a spectrogram, and tracking a high energy region in the spectrogram to form a trunk velocity contour on the spectrogram thereby obtaining the human body CSI gait parameter, wherein the trunk velocity contour is first human body velocity curve or highest energy curve.

In some embodiments, in the step (S5), to obtain the human body actual gait parameter and to make it a comparative standard to the human body CSI gait parameter, a step for obtaining the calibration equation comprises: fitting the human body CSI gait parameter with a human body actual gait parameter, wherein a step for obtaining the human body actual gait parameter comprises: equipping a mark on a first body part of the human body and tracking the mark using a tracking software thereby obtaining a first actual walking velocity curve. In some embodiments, the second human body velocity curve comprises the first actual walking velocity curve.

Embodiment 1: the specification of the transmitter (1) Tx and the receiver (2) Rx.

The present invention uses a Linux™ 802.11n CSI tool and its monitor mode to collect the CSI signal. Further, in the present invention, two minicomputers (GIGABYTE™ GB-BER7H-5800) are configured as the transmitter (1) and the receiver (2) respectively, wherein both the two minicomputers run Ubuntu 14.04 LTS system, and are equipped with Wi-Fi Link 5300 network interface controller (NICs) (Intel®), and three omnidirectional antennas to generate 9 of CSI data streams. Specifically, every CSI data stream comprises detailed measurement data of 30 subcarriers, and different frequency of signal composed of amplitude and phase. In addition, the microcomputers collect the data in the 5 GHz frequency band, specifically the central frequency is 5.32 GHz, the band width is 20 MHz, and the sampling rate is 1000 Hz. It is understood that the NIC can be used to capture the CSI signal and then transmit the CSI signal to the processor (3) thereby allowing the processor (3) to conduct subsequent signal processing.

Embodiment 2: the process of preprocessing of CSI signal.

The CSI signal obtained by the transmitter (1) Tx and the receiver (2) Rx comprises: a human body walking related signal and a noise coming from hardware and environment, to prevent the CSI signal from the interference of the noise, the CSI signal is preprocessed. Specifically, to obtain a denoised CSI signal, a band-pass filter is used to remove the high frequency part of noise and the affection of DC component in the CSI signal. Then, a principal component analysis (PCA) is performed on the denoised CSI signal to process the subcarrier from all the omnidirectional antennas and to reduce the dimensionality of denoised CSI signal, thereby obtaining a signal component related to the human gait or the walking mode.

Embodiment 3: the process of performing a feature extraction on the denoised CSI signal to obtain a human body CSI gait parameter.

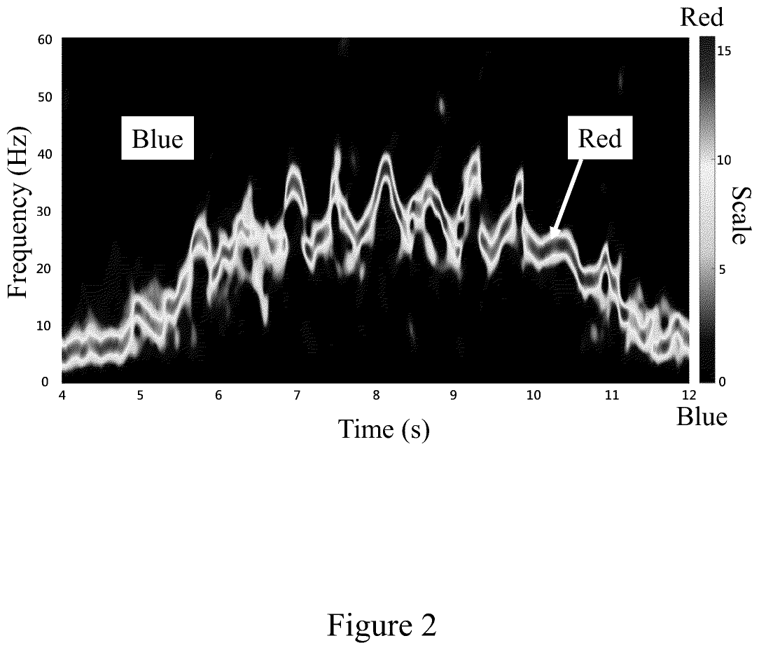

A short-time Fourier transform (STFT) is performed on the denoised CSI signal to change it into a spectrogram comprising an energy distribution, wherein the horizontal axis of the spectrogram is time, the vertical axis of the spectrogram is frequency. With reference to FIG. 2, the quality of the signal is improved by subtracting 2.5 times of standard deviation of frequency energy value at every time point for denoising.

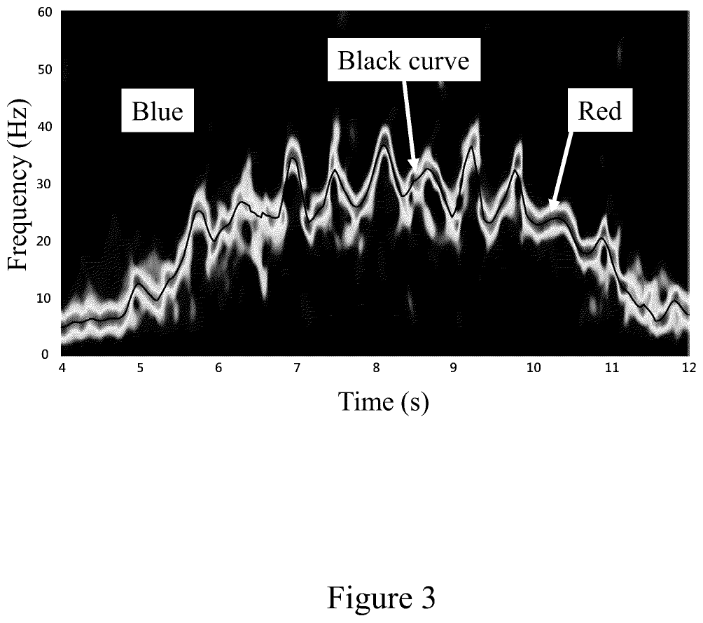

The following provides the method for obtaining a human walking velocity from the spectrogram: during the process of obtaining the CSI signal, the receiver (2) receives the signal reflected from the human body to analyze the gait of the human body, conceivably, the area of the trunk is largest, the largest quantity of the signal will be reflected from the trunk, thereby forming the most signal power shown in the spectrogram. As shown in FIG. 3, the black curve demonstrates that a human trunk moving velocity can be calculated according to a highest energy curve extracted from the contour of the highest energy band in the spectrogram, and a human body walking velocity can be derived from the human trunk moving velocity. Notably, the highest energy curve can be represented as the following equation: fₘₐₓ(t)=max{f|E(f,t)}.

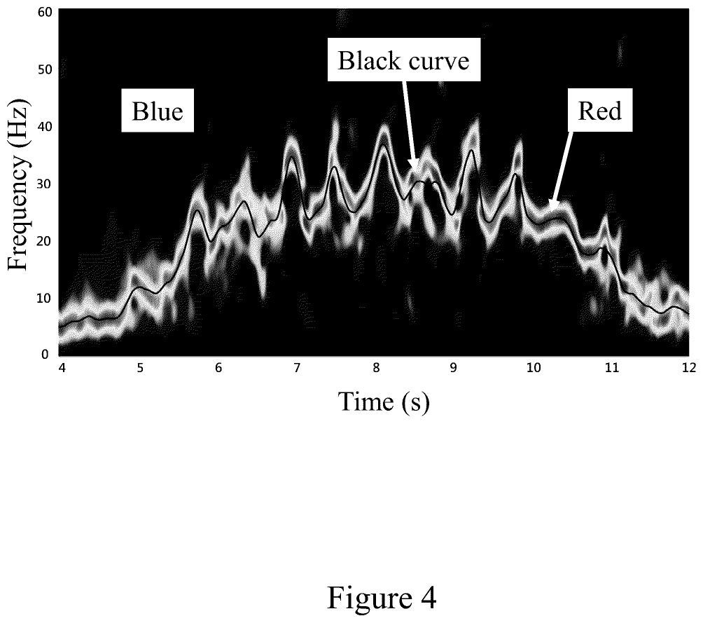

The following provides the method for obtaining a human body cadence from the spectrogram: providing a weighted average method: first, standardizing the energy value in the spectrogram to the range from 0 to 1; second, removing the frequency value where an energy strength is lower than 0.3; third, multiplying the frequency value at every time point by its energy strength to obtain a weighted average frequency f_{weighted}(t) as the following equation:

$${f}_{weighted} ( t ) = \{ \frac{1}{{E}_{total} ( t )} {\sum }_{i = 0}^{n} {E}_{i} ( t ) \times {f}_{i} ( t ) \vert {E}_{i} ( t ) > 0.3 \} ,$$

wherein fᵢ(t) represents the frequency at which the time is t, Eᵢ(t) represents the energy value of every fᵢ(t) in the spectrogram, Eₜₒₜₐₗ(t) represents the total energy at which the time is t, and variable n is the total number of frequencies satisfying the condition Eᵢ(t)>0.3. With reference to FIG. 4, compared with the highest energy curve, the black line, the weighted average curve of the human cadence, lacks the small fluctuations triggered by the noise coming from the environment, therefore provides stable and precise foundation for calculating the cadence.

Embodiment 4: the following provides a test using Wi-Fi and Tracker to demonstrate whether the walking velocity measured by the CSI reflects the actual walking velocity of the human body.

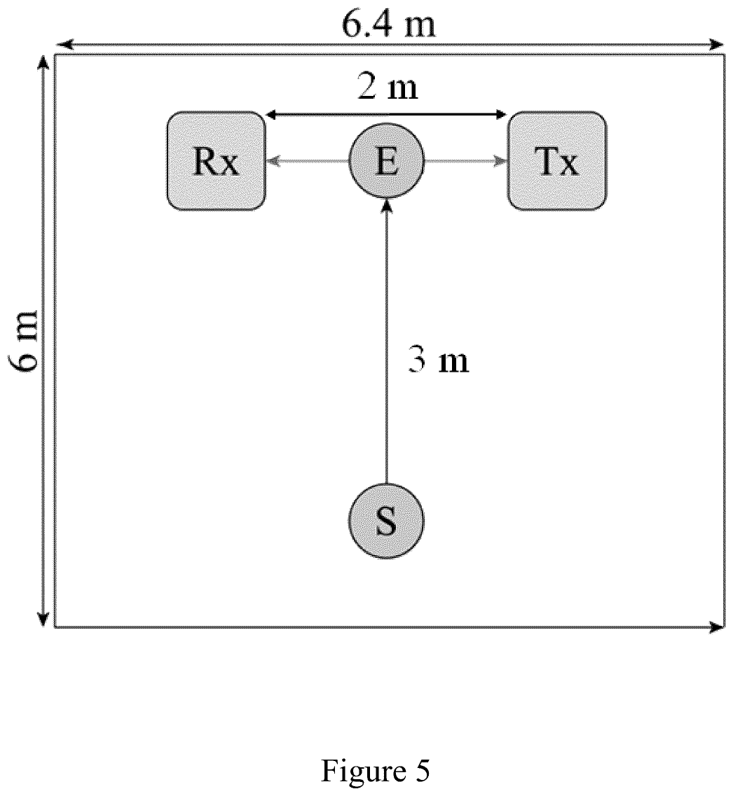

As shown in FIG. 5, first, in a vacant room (6.4 m long and 6.0 m wide), a transmitter (1) Tx and a receiver (2) Rx are arranged on a portable stand, and the line of sight (Los) between the transmitter (1) Tx and the receiver (2) Rx is adjusted to 2.0 m; second, allowing a subject (male, 180 cm, 68 kg) to walk along the perpendicular bisector of the Los from a start point (S) (3.0 m away from the Los) to an end point (E) (the midpoint of Los). Moreover, to make sure that the subject moves according to assigned cadence and step length, a metronome is used, and the floor is marked by colorful tape to show the assigned cadence and step length, thereby clearly guiding the subject.

From the start point (S) to the end point (E), the subject walks in any combination of three cadences (1 Hz, 1.5 Hz, and 2 Hz) and three step lengths (20 cm, 40 cm, and 60 cm). Notably, the condition (1.5 Hz+20 cm) and (2 Hz+20 cm) are excluded because they are impractical. The remaining 7 conditions and an additional condition that the subject walks in random cadences and step lengths (naturally), totally 8 conditions are tested, wherein every condition is tested for 10 times, totally 80 of CSI data fragments are collected, and a CSI velocity curve (the first human body velocity curve) is obtained.

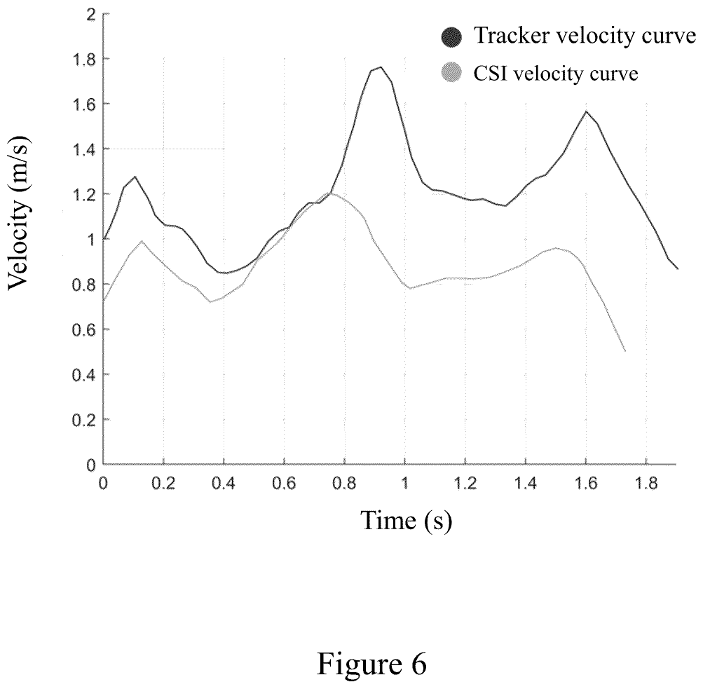

In this test, to obtain a Tracker velocity curve (the second human body velocity curve), a mark is equipped on the waist of the subject, then the subject is allowed to move according to the 8 conditions, and an image analyzing software Tracker, established by the Open Source Physics (OSP) Java, is used to track the mark on the waist of the subject and then calculate the actual walking velocity of the subject (VRA). As shown in FIG. 6, the difference showed by overlaying the human body actual walking velocity curve (Tracker velocity curve) obtained by using Tracker and the human body walking velocity curve (CSI velocity curve) obtained by using Wi-Fi demonstrates that, the CSI velocity curve shifted downward compared with the Tracker velocity curve, which means, the human body walking velocity measured using Wi-Fi is slower than the human body actual walking velocity, and a unignorable error exists therein.

Accordingly, the human body gait parameter measured using CSI needs to be calibrated to reflect the actual walking status of the human body. To determine whether the calibrated CSI velocity curve is close to the Tracker velocity curve, in this embodiment, a Dynamic Time Warping (DTW) is used to evaluate the similarity between the CSI velocity curve and the Tracker velocity curve, the smaller the DTW value, the higher the similarity between the two curves.

Embodiment 5: the establishment of the calibration equation

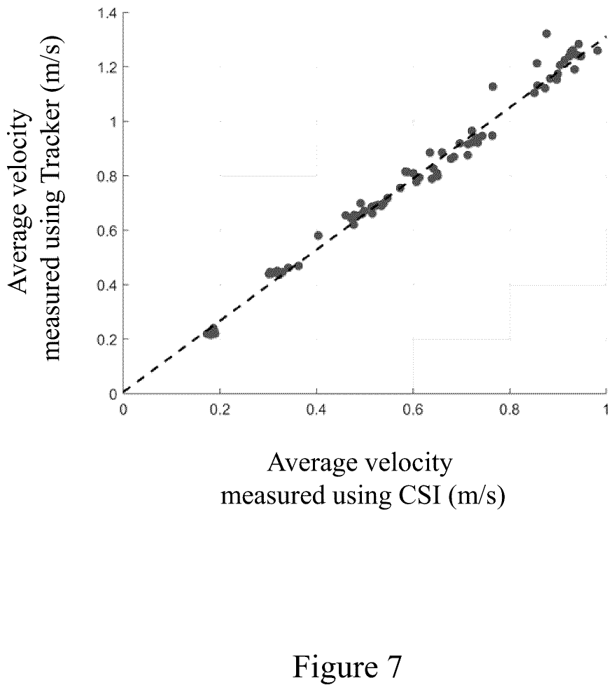

To eliminate the error between the CSI velocity curve and the Tracker velocity curve, to make the Wi-Fi-derived walking velocity closer to the human body actual walking velocity, and to realize a precise quantification of walking velocity accomplished without using a wearing device and an image monitoring device, the following provides a velocity quantification calibration function for calibrating the walking velocity measured by Wi-Fi. First, analyzing the 80 CSI data fragments to obtain an average walking velocity (V_{CSI}) derived from the CSI, and obtaining the human body actual walking velocity (V_{REAL}) measured by Tracker. Second, as shown in FIG. 7, a linear regression is performed to establish the correlation between the V_{CSI} and V_{REAL}, and a calibration equation can be derived: V_{correction}=1.305 V_{CSI}+0.006325. According to the analysis result: before the calibration, sum of squared errors (SSE) is 0.08533, R² is 0.9898; after the calibration, R² is 0.9897, root mean square error (RMSE) is 0.03307.

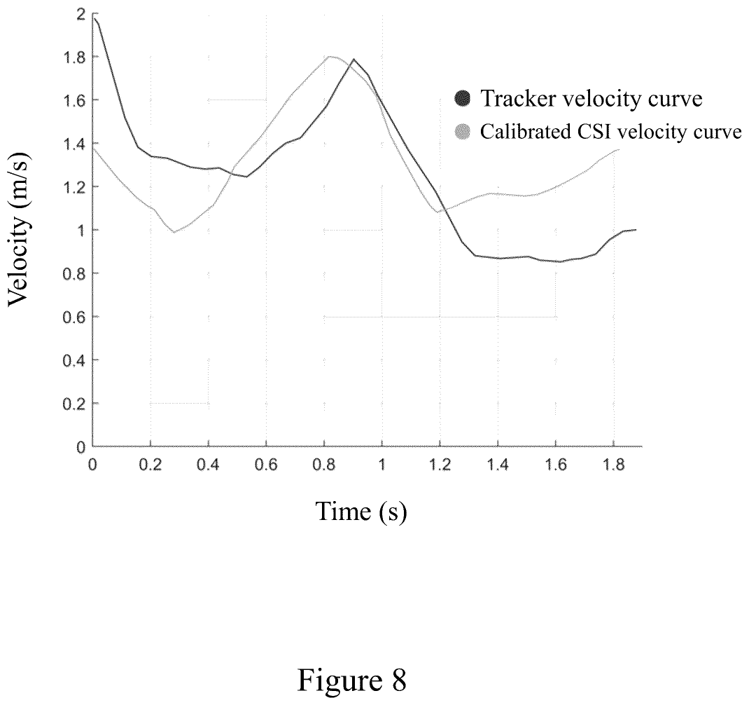

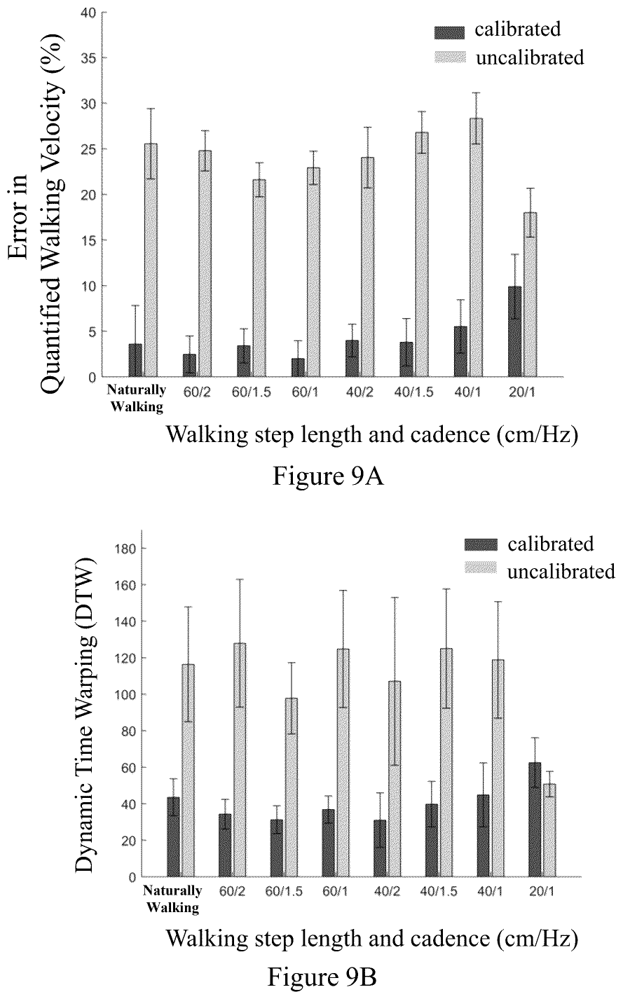

As shown in the calibration equation: V_{correction}=1.305 V_{CSI}+0.006325, wherein the coefficient is 1.305, the constant is 0.006325. According to the coefficient and the constant, it can be concluded as follows: (1) before calibrating the CSI velocity curve using the calibration equation, the CSI velocity curve is nearly completely lower than the Tracker velocity curve; (2) the range of extremum in the CSI velocity curve is smaller than in the Tracker velocity curve, indicating that the fluctuation of peak value and valley value is relatively smooth compared with the Tracker velocity curve; (3) the constant is small, indicating that the correlation between the CSI velocity curve and the Tracker velocity curve approximates a inclined line. As shown in FIG. 6, before calibration, the value in the CSI velocity curve is apparently smaller than the Tracker velocity, wherein the average error is approximately 25%, and the fluctuation is not significant, indicating that there is a difference between human body walking velocity measured by CSI and human body actual walking velocity; however, as shown in FIG. 8, after calibrating the CSI velocity curve using the calibration equation, the trajectory of change of calibrated CSI velocity curve is close to the Tracker velocity curve; further, as shown in FIG. 9A and FIG. 9B, after the calibration, the human body walking velocity (V_{correction}) measured using CSI is closer to the human body actual walking velocity (V_{REAL}), the average error between them is reduced to ≤5%, and DTW is reduced significantly, indicating that the human body walking velocity measured using CSI can truly reflect the human body actual walking velocity.

Embodiment 6: generalization tests will be provided as follows to estimate the performance of the calibration equation under different circumstances.

Multiple variables are arranged to be tested separately, the variable comprises: (1) individual differences of subjects, (2) relative correlation between the arrangements of transmitter (1) Tx and receiver (2) Rx, (3) relative position between walking path and the line formed by Los, (4) walking direction (toward or away from Los), walking angle (θ), length of Los (L), the distance the subject moves from the start point to the end point (D), and walking path offset (δ). On the other hand, other environments can be arranged to conduct the test, in some embodiments, the transmitter (1) Tx and receiver (2) Rx are arranged on both sides of the corridor, a Los between them is 1.6 m, the subject walks along the perpendicular bisector of the Los, and the subjects walks from the start point (3.6 m away from the Los) to the end point (0.8 m away from the Los).

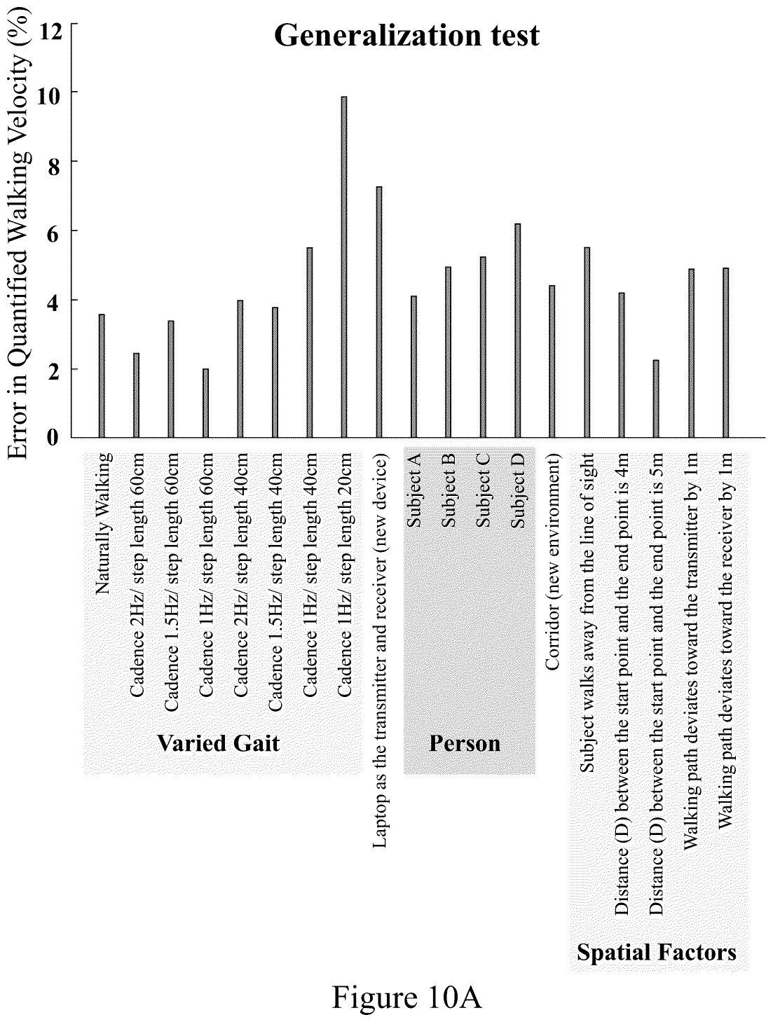

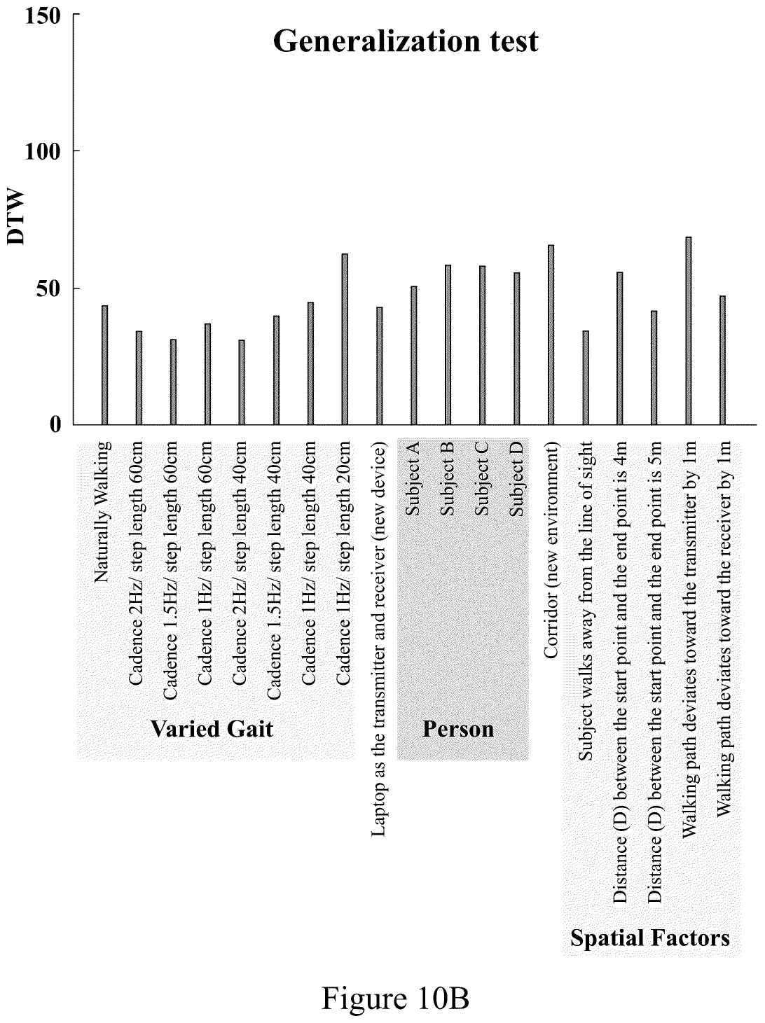

Table 1 concludes the test results after adjusting the variables, it should be found that the calibration equation can be generally used in groups with different variables. Specifically, as shown in FIG. 10A and FIG. 10B, when the subject moves with different cadences and different step lengths, the error of average walking velocity is 5%, and the value of DTW is 40; when different type of equipment are used as the transmitter (1) Tx and receiver (2) Rx, the error of average walking velocity rises to 7%, and the value of DTW is 43; when different subjects are tested, the average error stays within 5%, and the value of DTW is 55, when the test is conducted in the new environment (the corridor), the error of average walking velocity reduces to 4%, and the value of DTW rises to 65; when the walking direction is changed, the error of average walking velocity is 5%, and the value of DTW is 34, when the relative position of the line of the start point between the Los changes, the error of average walking velocity is 5%, and the value of DTW is 48; and when the walking path deviates from the perpendicular bisector of the Los, the error of average walking velocity is 5%, and the value of DTW is 58.

TABLE 1
The tested results under different variables

| | Value | |
|---|---|---|
| | The error of average | |
| Variables | walking velocity | DTW |
| Changes in cadence and different | 5% | 40 |
| step length | | |
| Different type of equipment as the | 7% | 43 |
| transmitter (1) Tx and receiver (2) | | |
| Rx | | |
| Different subjects | 5% | 55 |
| Different environment | 4% | 65 |
| Changing the walking direction | 5% | 34 |
| Changing the relative position of the | 5% | 48 |
| line of the start point between the | | |
| Los | | |
| The walking path deviates from the | 5% | 58 |
| perpendicular bisector of the Los | | |

Embodiment 7: the following describes a clinical research method for demonstrating the effectiveness of the present invention.

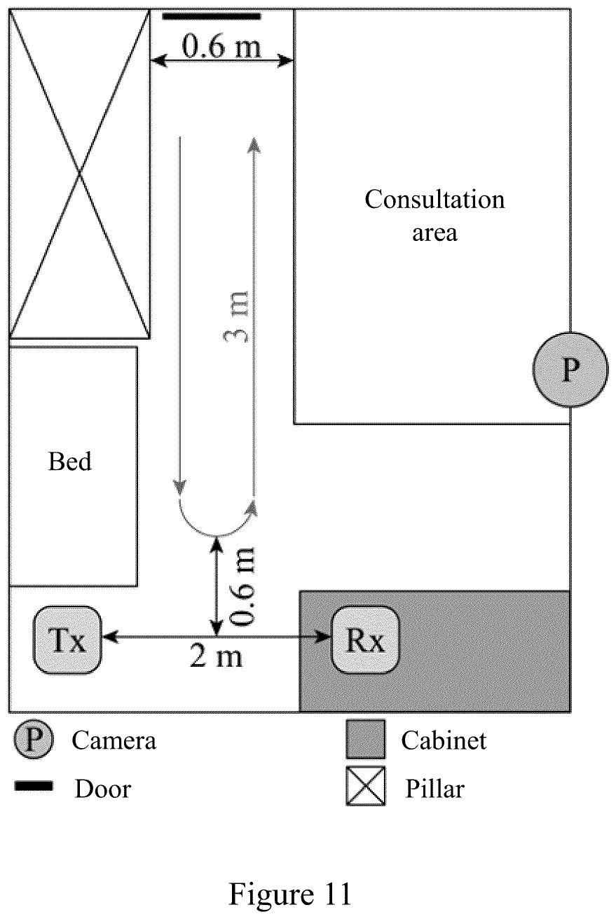

In this clinical research, 13 subjects of different ages, different sex, and different disease stage are tested in department of neurology, wherein 3 subjects are diagnosed with a Parkinson's disease. As illustrated in Table 2, in accordance with IRB protocol (NCKUH IRB, No. B-ER-112-076), the physician explains the details of this research during the clinical visit, the consent form for participating in the research and the use of data is provided by each subject, and the research is conducted under the surveillance of the physician wherein the subjects are receiving treatment. As shown in FIG. 11, FIG. 11 is a schematic diagram illustrating the arrangement of device in this research: the research is conducted in a checking room (4.5 m long, 3.6 m wide), the Los is 2.0 m between a transmitter (1) Tx and a receiver (2) Rx, and a GoPro® camera is configured to obtain a reference data for comparison.

TABLE 2
data collection protocol for clinical validation

| Mission | Operator(s) | Steps |
|---|---|---|
| Setting up for | device | 1. Tapes are applied on the start point |
| collecting | operator | and the corner point for providing |
| the data | | visual cues. |
| | | 2. Setting up the Wi-Fi device. |
| | | 3. Setting up the camera and ensuring |
| | | that its field of view covers the |
| | | entire area where the subjects walk. |
| Explaining | clinical | 1. Requesting that the subjects walk |
| the test | physician | as usual as they walk, instructing |
| | | the subjects the location of the start |
| | | point and the corner point, and |
| | | allowing the subjects to wear safety |
| | | belts. |
| | | 2. When hearing the instruction “now |
| | | you can walk”, instructing the |
| | | subjects to start walking. |
| | | 3. Requesting that the subjects wait |
| | | for the instruction “now you can |
| | | walk” in the start point. |
| Collecting | (a) device | 1. (a) starts using the camera to record. |
| the data | operator | 2. (a) starts recording the Wi-Fi data. |
| | (b) clinical | 3. (a) informs (b) that the arrangement |
| | physician | is complete, (b) should be able to |
| | | instruct the subjects to walk. |
| | | 4. (b) instructs the subjects “now you |
| | | can walk”. |
| | | 5. (b) instructs the subjects to walk |
| | | from the start point to the corner |
| | | point, then turn back to the start |
| | | point. |
| | | 6. (b) reminds the subjects to turn |
| | | around when the subject gets back to |
| | | the start point. |
| | | 7. (b) allows the subjects to repeat the |
| | | steps 4 to 6 times. |
| | | 8. (b) announces that the test is over, |
| | | and (a) stops the camera and the Wi- |
| | | Fi. |

Embodiment 8: the following provides the process of the clinical research of the Parkinson's patients.

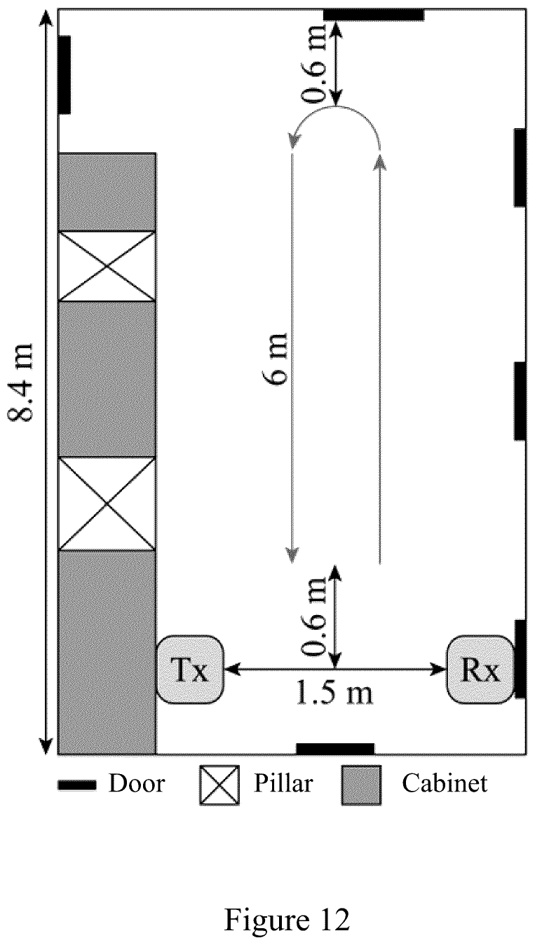

24 Parkinson's patients (subjects) comprising 12 males and 12 females are recruited, the age of the subjects range from 52 to 84 years old, the rating score of the unified Parkinson's Disease Rating Scale (UPDRS) of the subjects range from 0 to 2, wherein the rating score of 1 subject is 0, the rating score of 10 subjects is 1, and the rating score of 13 subjects is 2. As shown in FIG. 12, FIG. 12 is a schematic diagram illustrating the arrangement of device in this research: a transmitter (1) Tx and a receiver (2) are arranged in an end of a corridor, the Los is 1.5 m.

Embodiment 9: the following elaborates the factors that influence the accuracy of quantifying the gait parameter.

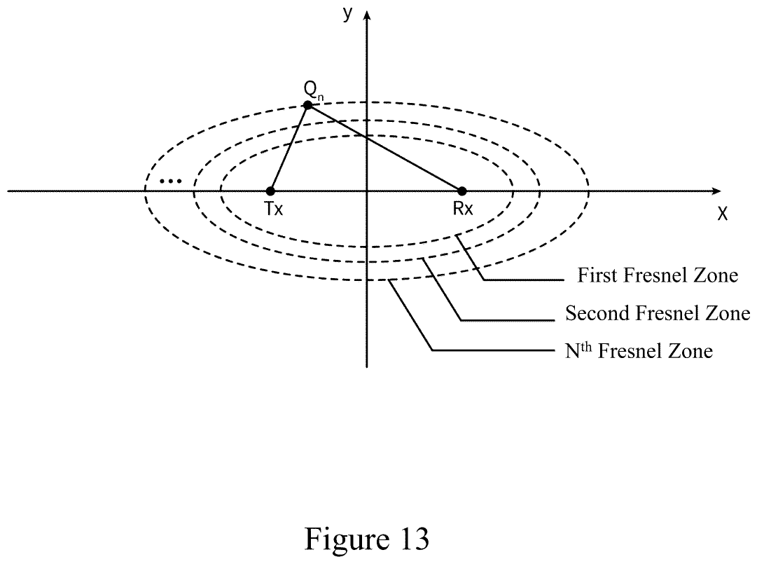

As shown in FIG. 13, the Fresnel zone theory:

$$\vert \overline{{T}_{X} {Q}_{n}} \vert + \vert \overline{{Q}_{n} {R}_{X}} \vert - \vert \overline{{T}_{X} {R}_{x}} \vert = \frac{n λ}{2} ,$$

wherein the n is a nature number, the Qₙ represents a point on an oval of the n Fresnel zone. according to the formula: when the difference between the length of reflection path (|T̅_̅{̅X̅}̅Q̅ₙ̅|+|Q̅ₙ̅R̅_̅{̅X̅}̅|) and the length of direct path (|T̅_̅{̅X̅}̅R̅ₓ̅|) is an odd multiple of half the wavelength, constructive interference occurs. The transmitter (1) Tx and the receiver (2) Rx are arranged on two common focusses of the oval of the n Fresnel zone respectively, and the direct path corresponds to the focal length of the oval, which is the distance between the transmitter (1) Tx and the receiver (2) Rx, the phenomenon indicates: when the direct path becomes longer, and the rate of change of the reflection path's length stays constant, the constructive interference reduces if the walking velocity of the human body stays constant, that causes the value of the human body gait parameter obtained by CSI to be lower than the value generated from actual walking process of the human body, therefore the actual walking velocity of the human body cannot be accurately reflected.

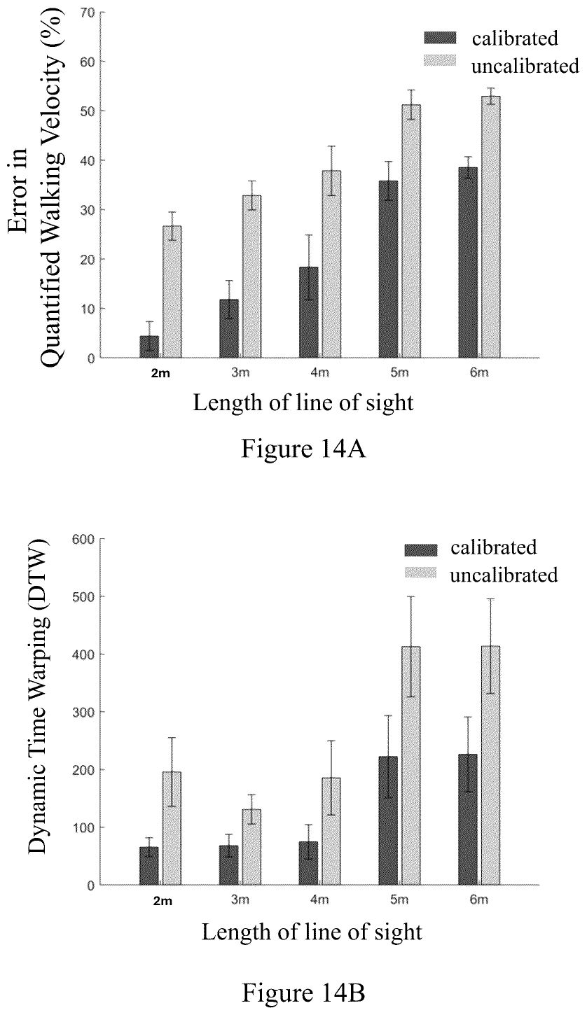

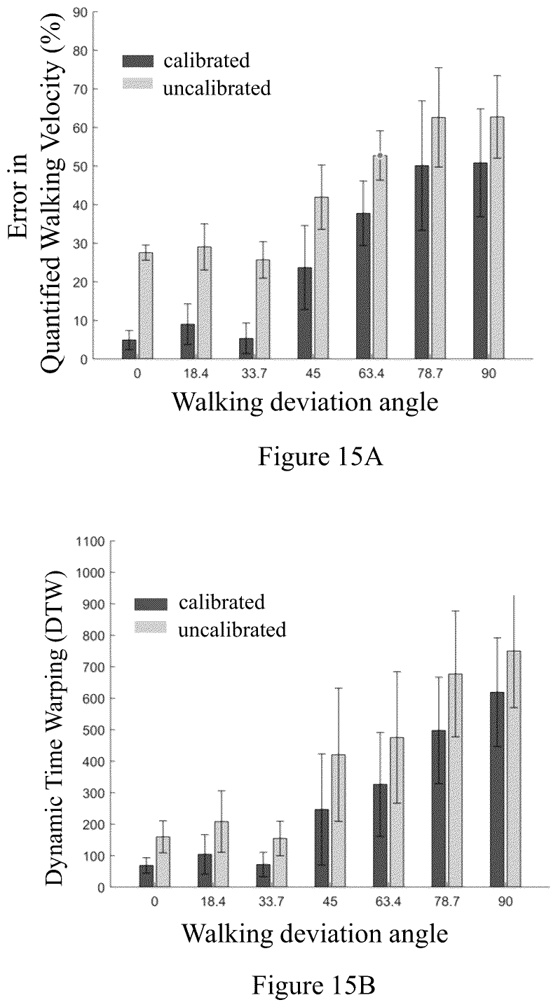

As shown in FIG. 14A and FIG. 14B, when the length of the Los reaches 6 m, before the calibration, average walking velocity measured by CSI (V_{CSI}) is only half of the human body average walking velocity (V_{REAL}), suggesting that the distance between the transmitter (1) Tx and the receiver (2) Rx has a significant influence on the accuracy of the human body gait parameter measured using Wi-Fi. On the other hand, the walking angle formed between the human body walking direction and the Los has a significant influence on the ability of Wi-Fi to quantify velocity. As shown in FIG. 15A to FIG. 15B, when the walking deviation angle is 0, representing that the subjects walk along the direction vertical to the Los, after the calibration, the error between the V_{CSI} and V_{REAL} is 3.6%; when the walking deviation angle is 90, representing that the subjects walk along the direction parallel to the Los, after the calibration, the error between the V_{CSI} and V_{REAL} still rises to 40%, the reason may comprises: under the condition that the walking velocity stays constant and the walking direction becomes parallel to the Los, the oval part of the Fresnel zone passed through by the subject reduces, thereby reducing the constructive interference under the same walking velocity and decreasing the quantitative value of the velocity.

In summary, through the test accomplished by diverse clinical cases, conclusions are provided as follows: under the same data preprocessing and the same test design, velocity quantification by using Wi-Fi is affected by: the length of the Los, the walking direction should be vertical to the Los, and the activity restriction of the subject in the environment. If the conditions aforementioned meet the standard, even the walking velocity of the Parkinsons's disease patient can be precisely quantified in the clinical environment, wherein the wearing device or the image monitor is not required during the quantification process, the gait parameter can be recorded more naturally, and the healthcare professionals or the caregiver will be informed when something wrong happens in the body of the patient.

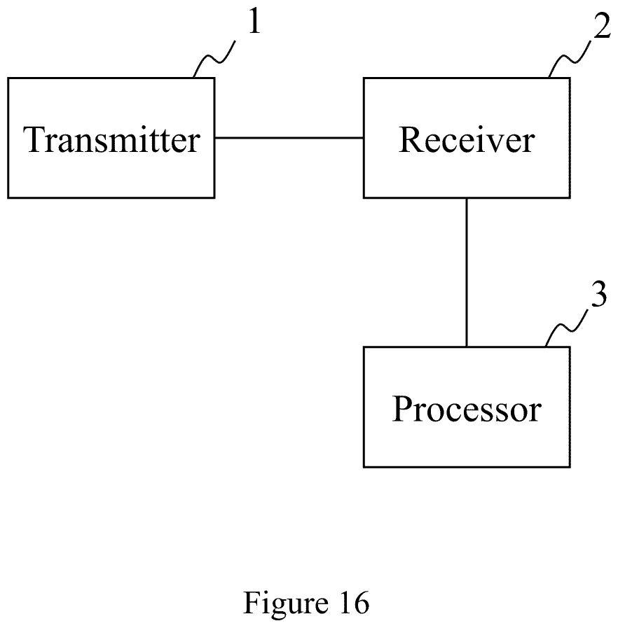

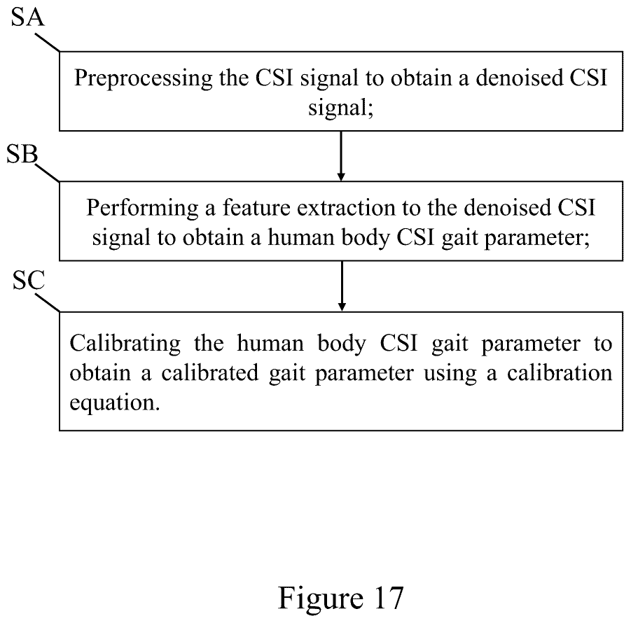

As shown in FIG. 16 and FIG. 17, the present invention further provides a system for health surveillance using Wi-Fi to quantify gait parameter, comprising: a transmitter (1) configured to emit a first wireless signal; a receiver (2) arranged relative to the transmitter to optionally receives a second wireless signal, from which a CSI signal is extracted, and the second wireless signal is formed by the first wireless signal reflected from a human body; and a processor (3) arranged relative to the receiver (2), wherein the processor (3) is configured to receive the CSI signal and execute an instruction as the following: (SA) preprocessing the CSI signal to obtain a denoised CSI signal; (SB) performing a feature extraction on the denoised CSI signal to obtain a human body CSI gait parameter; and (SC) calibrating the human body CSI gait parameter to obtain a calibrated gait parameter using a calibration equation. In some preferred embodiments, in the step (SC), a step for obtaining the calibration equation comprises: fitting the human body CSI gait parameter with a human body actual gait parameter, and establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

In some embodiments, the instruction further comprises: (SD) calculating T1 and T2 in one or more time period, and delivering a health surveillance report when T2>T1, wherein T1 is the total time in which the calibrated gait parameter (V_{correction}) of the subject is larger than the default threshold, T2 is the total time in which the calibrated gait parameter (V_{correction}) of the subject is smaller than the default threshold. In some embodiments, the instruction further comprises: (SE) calculating T1 and T2 in a first time period, wherein T1 is the total time in which the calibrated gait parameter (V_{correction}) of the subject is larger than the default threshold, T2 is the total time in which the calibrated gait parameter (V_{correction}) of the subject is smaller than the default threshold; calculating T3 and T4 in a second time period, wherein T3 is the total time in which the calibrated gait parameter (V_{correction}) of the subject is larger than the default threshold, T4 is the total time in which the calibrated gait parameter (V_{correction}) of the subject is smaller than the default threshold, and delivering a health surveillance report when

$$\frac{T 1}{T 1 + T 2} > \frac{T 3}{T 3 + T 4} ,$$

wherein the second time period is later than the first time period.

In some embodiments, to ensure the accuracy of the measurement, and to avoid the difference between the human body CSI gait parameter and the human body actual gait parameter is too large to calibrate, a line of sight A̅B̅ is formed between a point A on the transmitter (1) and a point B on the receiver (2), and 5 m≥A̅B̅≥1 m; and an angle θ is formed between a moving direction of the human body and a perpendicular bisector of the line of sight A̅B̅, and θ≤45°.

In some embodiments, to avoid interference of noise, and to prevent the accuracy of measurement of gait parameter from affecting by signal irrelevant to human gait, the step (SA) comprises: using a wave filter to filter a high frequency noise in the CSI signal, and performing a principal component analysis (PCA) on the CSI signal, thereby extracting a human body gait related signal from the CSI signal; or wherein the step (SB) comprises: performing a short-time Fourier transform (STFT) on the denoised CSI signal to obtain a spectrogram, and tracking a high energy region in the spectrogram to form a trunk velocity contour on the spectrogram thereby obtaining the human body CSI gait parameter.

In some embodiments, to obtain the human body actual gait parameter and to make it a comparative standard to the human body CSI gait parameter, in the step (SC), a step for obtaining the calibration equation comprises: fitting the human body CSI gait parameter with a human body actual gait parameter, wherein a step for obtaining the human body actual gait parameter comprises: equipping a mark on a first body part of the human body and tracking the mark using a tracking software thereby obtaining a first actual walking velocity curve.

The effectiveness of the present invention compared with prior art comprises: in prior art, there are gaps or differences between the human body gait measurement result measured by using Wi-Fi directly and the actual walking value of the human body, therefore the Wi-Fi measurement lacks practical value and cannot be used to determine a healthy degree of a subject. The method provided by the present invention comprises analyzing the result of CSI signal and fitting them with the human body actual gait parameter to obtain the calibration equation, wherein the gait parameter is close to the human body actual gait parameter after the gait parameter of CSI signal is calibrated by the calibration equation, thereby can be used to analyze the health status of the human body. On the other hand, the present invention can analyze one or more time period of a human body gait change curve, calculate the gait parameter to track the history measuring result of the subject's gait, and remind the subject when abnormality occurs in their body thereby improving the medication administration or the treatment strategy.

The foregoing description merely illustrates the preferred embodiments of the present invention and should not be construed as limiting the scope of the invention. Any simple equivalent changes or modifications made in accordance with the scope of the patent claims and the description of the invention shall fall within the scope covered by the patent of the present invention. Additionally, none of the embodiments or claims of the present invention are required to achieve all the objectives, advantages, or features disclosed herein. Furthermore, the abstract and titles are provided solely to facilitate patent documentation searches and are not intended to limit the scope of the invention. Moreover, terms such as “first” and “second” mentioned in the specification are used merely to denote the names of components and do not imply any upper or lower limit on the quantity of the components.

1. A method for health surveillance using Wi-Fi to quantify gait parameter, comprising:
(S1) using a transmitter and a receiver to establish a Wi-Fi space, wherein the transmitter is configured to emit a first wireless signal;
(S2) moving a human body in the Wi-Fi space to receive a second wireless signal by the receiver, and extracting a CSI signal from the second wireless signal, wherein the second wireless signal is formed by the first wireless signal reflected from the human body;
(S3) preprocessing the CSI signal to obtain a denoised CSI signal;
(S4) performing a feature extraction on the denoised CSI signal to obtain a human body CSI gait parameter; and
(S5) calibrating the human body CSI gait parameter using a calibration equation to obtain a calibrated gait parameter.

2. The method as claimed in claim 1, wherein
in the step (S1), a line of sight A̅B̅ is formed between a point A on the transmitter and a point B on the receiver, and 5 m≥A̅B̅≥1 m.

3. The method as claimed in claim 1, wherein
the step (S3) further comprises:
using a wave filter to filter a high frequency noise in the CSI signal.

4. The method as claimed in claim 1, wherein the calibration equation is obtained by a method comprising: fitting the human body CSI gait parameter with a human body actual gait parameter.

5. The method as claimed in claim 4, wherein the calibration equation is obtained by a method comprising: establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

6. A system for health surveillance using Wi-Fi to quantify gait parameter, comprising:
a transmitter configured to emit a first wireless signal;
a receiver arranged relative to the transmitter to optionally receive a second wireless signal and extract a CSI signal from the second wireless signal, wherein the second wireless signal is formed by the first wireless signal reflected from a human body; and
a processor arranged relative to the receiver and configured to receive the CSI signal and execute an instruction as the following:
(SA) preprocessing the CSI signal to obtain a denoised CSI signal;
(SB) performing a feature extraction on the denoised CSI signal to obtain a human body CSI gait parameter; and
(SC) calibrating the human body CSI gait parameter to obtain a calibrated gait parameter using a calibration equation.

7. The system as claimed in claim 6, wherein
a line of sight A̅B̅ is formed between a point A on the transmitter and a point B on the receiver, and 5 m≥A̅B̅≥1 m.

8. The system as claimed in claim 6, wherein
the step (SA) further comprises:
using a wave filter to filter a high frequency noise in the CSI signal; and
performing a principal component analysis (PCA) on the CSI signal, thereby extracting a human body gait related signal from the CSI signal.

9. The system as claimed in claim 6, wherein the calibration equation is obtained by a method comprising: fitting the human body CSI gait parameter with a human body actual gait parameter.

10. The system as claimed in claim 9, wherein the calibration equation is obtained by a method comprising: establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

11. The method as claimed in claim 2, wherein in the step (S2), an angle θ is formed between a moving direction of the human body and a perpendicular bisector of the line of sight A̅B̅, and θ≤45°.

12. The method as claimed in claim 3, wherein the step (S3) further comprises: performing a principal component analysis (PCA) on the CSI signal, thereby extracting a human body gait related signal from the CSI signal.

13. The method as claimed in claim 1, wherein the step (S4) further comprises: performing a short-time Fourier transform (STFT) on the denoised CSI signal to obtain a spectrogram.

14. The method as claimed in claim 13, wherein the step (S4) further comprises: tracking a high energy region in the spectrogram to form a trunk velocity contour on the spectrogram thereby obtaining the human body CSI gait parameter.

15. The method as claimed in claim 4, wherein the human body actual gait parameter is obtained by a method comprising: equipping a mark on a first body part of the human body and tracking the mark using a tracking software, thereby obtaining a first actual walking velocity curve.

16. The method as claimed in claim 15, wherein the calibration equation is obtained by a method comprising: establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.

17. The system as claimed in claim 7, wherein an angle θ is formed between a moving direction of the human body and a perpendicular bisector of the line of sight A̅B̅, and θ≤45°.

18. The system as claimed in claim 8, wherein the step (SB) further comprises: performing a short-time Fourier transform (STFT) on the denoised CSI signal to obtain a spectrogram; and tracking a high energy region in the spectrogram to form a trunk velocity contour on the spectrogram, thereby obtaining the human body CSI gait parameter.

19. The system as claimed in claim 9, wherein the human body actual gait parameter is obtained by a method comprising: equipping a mark on a first body part of the human body and tracking the mark using a tracking software, thereby obtaining a first actual walking velocity curve.

20. The system as claimed in claim 19, wherein the calibration equation is obtained by a method comprising: establishing the correlation between the human body CSI gait parameter and the human body actual gait parameter using a linear regression to obtain the calibration equation: V_{correction}=1.305 V_{CSI}+0.006325, wherein the V_{CSI} is a human body walking velocity obtained by analyzing the CSI signal, the V_{correction} is a calibrated human body walking velocity obtained by calibrating the V_{CSI}.
